(19) United States
(12) Patent Application Publication
(10) Pub. No.: US 2025/0182045 A1
(43) Pub. Date: Jun. 5, 2025

(54) ASSIGNMENT OF ENVIRONMENTAL ATTRIBUTES IN PRODUCTION NETWORKS

(71) Applicant: BASF SE, Ludwigshafen (DE)

(72) Inventors: Ana ALBA PEREZ, Ludwigshafen (DE); Kurt WENZL, Ludwigshafen (DE)

(21) Appl. No.: 18/723,376

(22) PCT Filed: Dec. 19, 2022
(86) PCT No.: PCT/EP2022/086655

§ 371 Date: Jun. 21, 2024

Related U.S. Application Data
(60) Provisional application No. 63/416,091, filed on Oct. 14, 2022.

(30) Foreign Application Priority Data
Dec. 21, 2021 (EP) 20216326.5
Dec. 21, 2021 (EP) 20216327.3
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06Q 10/087

(52) U.S. Cl. CPC G06Q 10/087

(57) ABSTRACT

Disclosed are methods, apparatuses and systems for producing at least one chemical product associated with one or more environmental attribute(s) and for assigning at least one environmental attribute to at least one chemical product produced by a chemical production network.

### TECHNICAL FIELD

The present disclosure relates to methods, apparatuses and systems for producing at least one chemical product associated with one or more environmental attribute(s) and for assigning at least one environmental attribute to at least one chemical product produced by a chemical production network.

### TECHNICAL BACKGROUND

In supply chains the environmental impact of each supply chain participants is of great interest. Transparency between the participants can aid collective reduction of environmental impacts to combat climate change. However, data sharing of environmental impact data is hindered by the lack of common data standards and the lack of trusted data platforms. In addition, the highly specific and centralized setup of data systems today makes exchange and sharing for collective action laborious. Hence, there is a need to develop metrices quantifying the environmental impact of produced products, to simplify data standards relating to environmental impact and to broadly enable a secure exchange of supply chain data relating to the environmental impact.

### SUMMARY OF THE INVENTION

In one aspect a method for producing at least one chemical product associated with one or more environmental attributes, wherein the chemical product is produced by a chemical production network, the method comprising:
providing one or more input material(s) associated with one or more environmental attribute(s) to the chemical production network,
providing at least one balancing account associated with one or more environmental attribute(s) of the one or more input material(s) provided to the chemical production network,
producing the at least one chemical product,
providing an identifier associated with the chemical product,
assigning at least one environmental attribute from the at least one balancing account associated with the respective environmental attribute to the at least one chemical product, wherein the environmental attribute from the balancing account is assigned to the identifier associated with the chemical product.

In another aspect a method for assigning at least one environmental attribute to at least one chemical product produced by a chemical production network, the method comprising:
providing at least one balancing account associated with one or more environmental attribute(s) and an account balance associated with one or more environmental attribute(s) from one or more input material(s) provided to the chemical production network,
providing an identifier associated with the chemical product.
assigning at least one environmental attribute from the at least one balancing account associated with the respective environmental attribute to the at least one chemical product, wherein the environmental attribute from the balancing account is assigned to the identifier.

In another aspect an apparatus for assigning at least one environmental attribute to at least one chemical product produced by a chemical production network, the method comprising:
an accounting system configured to provide at least one balancing account associated with one or more environmental attribute(s) and an account balance associated with one or more environmental attribute(s) from one or more input material(s) provided to the chemical production network,
an identifier provider configured to provide an identifier associated with the chemical product,
an assignor configured to assign at least one environmental attribute from the at least one balancing account associated with the respective environmental attribute to the at least one chemical product, wherein the environmental attribute from the balancing account is assigned to the identifier associated with the chemical product.

In another aspect a system for producing at least one chemical product associated with one or more environmental attributes, wherein the chemical product is produced by a chemical production network, the system comprising:
a chemical production network configured to produce at least one chemical product based on one or more input material(s) provided to the chemical production network, wherein the one or more input material(s) are associated with one or more environmental attribute(s);
an accounting system configured to provide at least one balancing account associated with one or more environmental attribute(s) of the one or more input material(s) provided to the chemical production network,
an identifier provider configured to provide an identifier associated with the chemical product,
an assignor configured to assign at least one environmental attribute from the at least one balancing account associated with the respective environmental attribute to the at least one chemical product, wherein the environmental attribute from the balancing account is assigned to the identifier associated with the chemical product.

In yet another aspect disclosed is a computer element, in particular a computer program product or a computer readable medium, with instructions, which when executed by one or more processor(s) are configured to carry out the steps of any of the methods disclosed herein. In yet another aspect disclosed is a computer element, in particular a computer program product or a computer readable medium, with instructions, which when executed by one or more processor(s) cause any of the apparatuses disclosed herein to perform any of the methods disclosed herein.

Disclosed is in yet another aspect the use of the chemical product associated with one or more environmental attribute(s) as provided by any of the methods disclosed herein and/or produced by a chemical production network as provided by any of the methods disclosed herein to produce at least one discrete product or at least one end product associated with the one or more environmental attribute(s). The at least one discrete product or the at least one end product may be an intermediate or end product of a product supply chain. The at least one discrete product or the at least one end product may be based on one or more target material(s). The at least one discrete product or the at least one end product may be produced by discrete manufacturing. Disclosed is in yet another aspect a method for producing at least one discrete product or at least one end product associated with the one or more environmental attribute(s), wherein the chemical product associated with one or more environmental attribute(s) as provided by any of the methods disclosed herein and/or produced by a chemical production network as provided by any of the methods disclosed herein is provided and/or used to produce the at least one discrete product or at least one end product associated with the one or more environmental attribute(s).

Disclosed is in yet another aspect the use of the one or more environmental attribute(s) assigned to the chemical product identifier as provided by any of the methods disclosed herein to provide at least one discrete product identifier relating to at least one discrete product or at least one end product identifier relating at least one end product to the one or more environmental attribute(s). Disclosed is in yet another aspect a method for providing at least one discrete product identifier relating at least one discrete product or at least one end product identifier relating at least one end product to the one or more environmental attribute(s), wherein the one or more environmental attribute(s) assigned to the chemical product identifier as provided by any of the methods disclosed herein are provided and/or used to provide the at least one discrete product identifier relating at least one discrete product or at least one end product identifier relating at least one end product to the one or more environmental attribute(s).

### EMBODIMENTS

Any disclosure and embodiments described herein relate to the methods, the apparatuses, the computer elements and uses lined out above or below and vice versa. The benefits provided by any of the embodiments and examples equally apply to all other embodiments and examples and vice versa.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

The methods, apparatuses, systems and computer elements disclosed herein provide an efficient way to track environmental attributes in chemical processing and to provide chemical products with positive environmental impact through the value chain. By using an identifier concept environmental attributes associated with input materials can be efficiently assigned to chemical products produced in chemical productions networks. Specifically for chemical networks that produce more than one chemical product from more than one input material via interconnected, connected and non-connected production chains, the use of the identifier concept for chemical products allows to reliably assign environmental attributes in line with the physical setup of the chemical production network. This further allows to decouple the complexity in material flow of chemical production networks while still allowing to assign environmental impact to chemical product. This way the environmental impact of the produced chemical product can be determined in line with the physical set up of the chemical production network. Moreover, the environmental property of the chemical products produced by the chemical production network can be made transparent to customers further processing the chemical products. By providing chemical product identifiers associated with at least one environmental attribute or chemical product identifiers and environmental attribute identifier(s), the environmental attributes may even be adjusted to customer needs.

Environmental attributes may refer to a property or characteristic related to the environmental impact. Such property may be a property or characteristic of the input materials or chemical products. The environmental attribute may indicate an environmental performance of one or more material(s). The environmental attribute may be produced from properties of the input materials, the chemical production network and/or the target materials. The environmental attribute may be associated with the environmental impact of one or more material(s) at any stage during the lifecycle. The stages of the material or product lifecycle may include the stages of providing raw material, producing products, such as intermediate products or end products, using products, treating end-of-life products, recycling end-of-life products, disposing end-of-life products, reusing components from end-of-life products or any subset of stages. The environmental attribute may be specified or may be produced from any activity of one or more entities participating at any stage of the lifecycle of one or more material(s) or product(s).

The environmental attribute may include one or more characteristic(s) that are attributable to environmental or sustainability impact of the material or product. The environmental attribute may include environmental, technical, recyclability, circularity or complementary risk characteristics(s), characteristic(s) associated with the environmental impact of one or more material(s) or product(s).

Environmental characteristic(s) may specify or quantify ecological criteria associated with the products environmental impact. Environmental characteristic(s) may be or may be produced or derived from measurements taken during the lifecycle of one or more product(s). Environmental characteristics may be determined at any stage of the product lifecycle and may characterize the environmental impact of the product for such stage or up to such stage. Environmental characteristic(s) may for example include impact categories such as carbon footprint, greenhouse gas emissions or global warming potential, primary energy demand, cumulative energy demand, biotic and abiotic resource consumption, air emissions, stratospheric ozone depletion potential, ozone formation, terrestrial and/or marine acidification, water consumption, water depletion, water availability, water pollution, noise pollution, freshwater and/or marine eutrophication potential, human carcinogenic and/or non-carcinogenic toxicity, photochemical oxidant formation, particulate matter formation, terrestrial, freshwater and/or marine ecotoxicity, ionizing radiation, agricultural and/or urban land occupation, land transformation, land use, indirect land use, deforestation, biodiversity, mineral resource consumption, fossil resource consumption. Environmental characteristic(s) may be calculated from combinations of one of more environmental characteristics. Environmental characteristic(s) may for example include product or material characteristics related to the production of the material or product like renewable, bio based, vegan, halal, kosher, palm oil-free, natural or the like.

Technical characteristic(s) may specify or quantify product performance at least indirectly associated with the environmental impact. Technical characteristic(s) may be or may be produced from measurements taken during the lifecycle of one or more product(s). Technical characteristics may be determined at any stage of the product lifecycle and may characterize the product performance for such stage or up to such stage. Technical characteristic(s) may for example include product composition data, input in the production process, bill of materials, product specification data, product component data, product safety data, application property data, application instructions or product quality data. Technical characteristic(s) may for example include physical, chemical or further properties of the material or product.

Circularity characteristic(s) may specify or quantify the products life cycle characteristics associated with circular uses. Circularity characteristic(s) may be or may be produced from measurements taken during the lifecycle of one or more product(s). Circularity characteristic(s) may be or may be produced from circular data recorded in one or more prior lifecycle(s) including reuse. Circularity characteristics may be determined at any stage of the product lifecycle and may characterize the reuse or recycling performance for such stage or up to such stage. Circularity characteristic(s) may relate to technical, mechanical, chemical and/or biological recycling. Circularity characteristic(s) may for example include recycling data, reuse rate, recycling rate, recycling loops, durability, utility during the use phase, destination after use, reuse reused product performance, reused product quality or the like. Further circularity material characteristics may be derived by combining circularity characteristic(s).

Recyclability characteristic(s) may specify or quantify the material or product life cycle characteristics associated with recycling uses. Recyclability characteristic(s) may include the composition of the material including specifically tailored constituents making the material suitable for recycling. Recyclability characteristic(s) may be or may be produced from measurements taken during the lifecycle of one or more materials or product(s). Recyclability characteristic(s) may be or may be produced from recycling data recorded in one or more prior lifecycle(s). Recyclability characteristics may be determined at any stage of the material or product lifecycle and may characterize the recycling performance for such stage or up to such stage. Recyclability characteristic(s) may for example include recycling data, recyclability data, efficiency of recycling, or the like.

Complementary risk characteristic(s) may specify or quantify characteristics associated with further sustainability related risks associated with the product at all stages. Complementary risk characteristic(s) may include the compliance with laws and/or voluntary standards, compliance with human rights declaration, reporting of sustainability development goals or the like. Complementary risk characteristic(s) may include material price variation, supply risks, obsolescence risk, food-contact compatibility or the like.

Chemical production networks may include multiple types of production processes for producing different chemical products from input materials. The chemical production network may include a complex production network producing multiple chemical products in multiple production chains. The chemical production network may include connected, interconnected and/or non-connected production chains. The chemical production network may produce from input materials multiple intermediates and from intermediates chemical products. Input material may enter the chemical production network at entry points. Chemical product may leave the production network at exit points.

The chemical production network may comprise one or more entry points at which input materials are provided to the chemical production network. Input material may include fossil material, non-fossil material or both. Fossil input material may include crude oil, natural gas, coal, or derivates of those. Non fossil input material may include renewable material, bio-based material or recycled materials. Input material may include feedstock for a gasification plant, a steam cracker or synthesis gas plant. Input material may include synthesis gas produced from fossil feedstock, non-fossil feedstock or both. Input material may include for example pyrolysis oil from recycled waste, syngas produced from recycled waste, naphtha produced from bio-based material, methane from bio-based material, or combinations thereof. Input material may be provided to at least one gasification plant, steam cracker or synthesis gas plant, or any plant of the production chain for downstream products such as nitrogen, ammonia, methanol, ethylene, propylene, sulfur or the like. Input material may include intermediate chemical products produced elsewhere with fossil and/or non-fossil input materials. Input material may include anorganic materials with mineral origin, salts, metals, glass or the like.

The input material associated with one or more environmental attribute(s) provided to the entry point of the chemical production network may include recycled input materials including, but not limited to, recycled pyrolysis oil, recycled pyrolysis gas, recycled synthesis gas, recycled hydrogen, recycled naphtha, recycled methane, recycled ethane, recycled propane, recycled chemicals or combinations thereof. Recycled chemicals may include, but may not be limited to, recycled ammonia, recycled methanol, recycled ethylene, recycled propylene, recycled benzene, recycled toluene, recycled xylene or combinations thereof. Recycled chemicals may include, but may not be limited to, recycled polymers, recycled oligomers, recycled monomers, or combinations thereof. In the context provided here recycled input material may include any material that at least in part includes recycled content and/or is at least in part produced from recycled content. The recycled content may be but does necessarily have to be physically and/or chemically traceable,

The input material associated with one or more environmental attribute(s) provided to the entry point of the chemical production network may include renewable input materials including, but not limited to, renewable pyrolysis oil, renewable pyrolysis gas, renewable synthesis gas, renewable hydrogen, renewable naphtha, renewable methane, renewable ethane, renewable propane, renewable chemicals or combinations thereof. Renewable chemicals may include, but may not be limited to, renewable ammonia, renewable methanol, renewable ethylene, renewable propylene, renewable benzene, renewable toluene, renewable xylene or combinations thereof. Renewable chemicals may include, but may not be limited to, renewable polymers, renewable oligomers, recycled monomers or combinations thereof. In the context provided here renewable input material may include any material that at least in part includes renewable content and/or is at least in part produced from renewable content. The renewable content may be but does necessarily have to be physically and/or chemically traceable.

The input material associated with one or more environmental attribute(s) provided to the entry point of the chemical production network may include bio-based input materials including, but not limited to, bio-based pyrolysis oil, bio-based pyrolysis gas, bio-based synthesis gas, bio-based hydrogen, bio-based naphtha, bio-based methane, bio-based ethane, bio-based propane, bio-based chemicals or combinations thereof. Bio-based chemicals may include, but may not be limited to, bio-based ammonia, bio-based methanol, bio-based ethylene, bio-based propylene, bio-based benzene, bio-based toluene, bio-based xylene or combinations thereof. Bio-based chemicals may include, but may not be limited to, bio-based polymers, bio-based oligomers, bio-based monomers or combinations thereof. In the context provided here bio-based input material may include any material that at least in part includes bio-based content and/or is at least in part produced from bio-based content. The bio-based content may be but does necessarily have to be physically and/or chemically traceable.

The chemical production network may include identity preserving or segregated production chains. Identity preserving or segregated in this context may refer to the environmental attributes of the input materials being preserved or segregated in the production chains. Examples are bio-based-renewable or recycled input materials used to produce the chemical product without fossil content. Further examples are fossil input materials used to produce the chemical products with fossil content. Chemical production networks may include non-identity preserving or non-segregated production chains. Non-identity preserving or non-segregated in this context may refer to the environmental attributes of the input materials being mixed in the production chains. For example, non-identity preserving or non-segregated in this context refers to the environmental attributes of the input materials being mixed with fossil input materials in the production chains. Examples are fossil and renewable input materials mixed to produce the chemical product with fossil and renewable content.

The chemical production network may include multiple production steps for one or more production chains. The production steps included in the chemical network may be defined by the physical system boundary of the chemical production network. The system boundary may be defined by location or control over production processes. The system boundary may be defined by the site of the chemical production network. The system boundary may be defined by production processes controlled by one entity or multiple entities jointly. The system boundary may be defined by value chain with staggered production processes to an end product, which may be controlled by multiple entities separately. The chemical production network may include a waste collection step, a waste sorting step, a recycling step such as chemical recycling through pyrolysis, a cracking step such as steam cracking, a separation step to separate outputs of one process step and further processing steps to convert such outputs to chemical products leaving the system boundary of the chemical production network. The entry points of the chemical production network may be marked by the entry of input materials to the chemical production network. The input materials entering the chemical production network may be used to produce one or more chemical products. The chemical products may leave the physical system boundary of the chemical production network. The exit points of the chemical production network may be marked by the exit of chemical products from the chemical production network.

The virtual balancing account may refer to a digital storage structure that stores data related to environmental attributes. The account may be associated with meta data identifying the account for balancing environmental attributes. The account may be associated with meta data identifying the environmental attributes and the environmental units allocated to the account. The account may be associated with meta data identifying the production chain the account is associated with. The account may be associated with meta data identifying the input or target material the account is associated with. The account may be part of a balancing system including multiple accounts. The account may hold environmental attributes for transaction. Environmental attributes may be allocated, added, deleted, withdrawn or deducted from the account. The virtual balancing account may by associated with environmental attribute types such as recycled or renewable. The virtual balancing account may by associated with input material types such as pyrolysis oil, bio-naphta, bio-methane, or combinations thereof. The virtual balancing account associated with the environmental attribute type recycled may be further associated with waste-stream type such as mixed plastics waste, specific end product waste, e.g. tiers waste or foam waste, post-consumer waste, pre-consumer waste or combinations thereof. The virtual balancing account may by associated with an allocation scheme such as segregated allocation, non-segregated allocation such as book and claim, mass balance with free attribution, mass balance without free attribution or combinations thereof.

The chemical product may be produced by the chemical production network to which the input material(s) associated with one or more environmental attribute(s) were provided. The chemical product may be produced by a production chain of the chemical production network to which the input material(s) associated with one or more environmental attribute(s) were provided. The chemical product may be produced from the input material(s) associated with one or more environmental attribute(s).

The identifier associated with the chemical product may include one or more identifier(s) relating to the chemical product. The identifier may relate to a chemical product class, a specific chemical product and/or properties of the chemical product such as environmental properties. The identifier may include a unique number uniquely associated with the chemical product class, the specific chemical product and/or the properties of the chemical product. The identifier may include one or more specific identifier(s), such as chemical product class identifier, specific chemical product identifier and/or property of the chemical product identifier. Such specific identifier(s) may be uniquely linked to the chemical product. For example, one or more property identifier(s) may be uniquely linked to the chemical product identifier. The chemical product identifier may be uniquely linked to the specific chemical product. This way the chemical product can be uniquely linked to a digital twin of the chemical product specifying specific properties of the chemical product.

The identifier associated with the chemical product may include one or more identifier(s) relating to one or more environmental attribute(s). The identifier may include an environmental attribute identifier, such as an unique environmental attribute identifier, relating to environmental attribute(s) assignable to chemical products. The environmental attribute identifier may relate to the chemical product class or the specific chemical product. For example, the environmental attribute identifier may relate to recycled content, bio-based content and/or renewable content as environmental attribute, each having their own unique each having their own unique material identifier. The specific environmental attribute or a specific combination of environmental attributes may be related to the unique environmental attribute identifier.

The identifier may include, be linked to or be related to a batch and/or order number, such as an unique batch and/or order number. The batch number may be linked to the physical entity of produced chemical product batches. The order number may be linked to the transaction specifying the shipment of the chemical product batch from the producer of the chemical product to the user further processing the chemical product.

In one embodiment the identifier is a chemical product identifier relating to a chemical product class and/or a specific chemical product including one or more environmental attribute(s). The identifier may include a chemical class identifier, such as unique class identifier, relating to a chemical product class. Chemical product classes may include chemical products grouped into chemical product classes. A class of chemical products (also referred to as a chemical product class) may include a broad specification classification that contains specific chemical products. The class of chemical products may be related to a unique class identifier (e.g., unique class number or other type of unique identifier). An example class is thermoplastics. The class thermoplastics may be related to the unique class identifier. The identifier may include a chemical product identifier, such as an unique material identifier, relating to a specific chemical product e.g. of a chemical product class. For example, the thermoplastics class may encompass numerous specific (unique) thermoplastics, each having their own characteristics. The specific thermoplastics may be related to the unique material identifier (e.g., unique material number or other type of unique identifier). For example, specific (unique) thermoplastics may be polyethylene, polystyrene, polypropylene, each having their own unique material identifier.

In another embodiment the chemical product identifier relates to a pre-defined chemical product specification and/or one or more pre-defined environmental attribute(s). Chemical products and chemical product specifications, such as composition, toxicological properties and ecotoxicological properties, are subject to regulated requirements and need to be approved by regulatory bodies. Owing to such approval process, not only new but also changes in existing chemical product specification may require approval process prior to marketing. The chemical product identifier relating to the chemical product may hence be pre-defined for specific chemical products having approved chemical product specification. The chemical product specification may include as composition, toxicological properties and ecotoxicological properties of the chemical product. The chemical product specification may require approval process prior to marketing.

Similarly chemical product(s) associated with one or more environmental attribute(s) may be subject to regulated requirements and need to be approved and registered with regulatory bodies. The chemical product specification may include one or more pre-environmental attribute(s) associated with the chemical product. The chemical product specification may require approval process prior to marketing. The chemical product identifier relating to the chemical product and one or more pre-defined environmental attribute(s) associated with the chemical product may hence be pre-defined for specific chemical products having approved chemical product specification.

In another embodiment the chemical product identifier relates to a pre-defined chemical product specification and one or more pre-defined set(s) of one or more environmental attribute(s). The chemical product identifier may relate to pre-defined set(s) of one or more environmental attribute(s) associated with the pre-defined for specific chemical products having approved chemical product specification. The pre-defined set of one or more environmental attribute(s) may include environmental attributes relating to technical, recyclability or circularity characteristics(s) associated with the environmental impact of chemical product(s). The pre-defined set of one or more environmental attribute(s) may relate to recycled content, renewable content, bio-based content, renewable energy, carbon capture, carbon utilization, heat integration or combinations thereof. The pre-defined set of one or more environmental attribute(s) may specify one or more environmental characteristic(s) including environmental, technical, recyclability or circularity characteristics(s) associated with the environmental impact of chemical product(s) and their production.

By pre-defining the chemical product specification and/or one or more pre-defined environmental attribute(s) for the chemical product identifier, a reliable and robust management of chemical products and associated environmental attributes can be provided. Through the pre-defined structure the assignment of identifiers is to chemical products and associated environmental attributes can be simplified and streamlined. This way chemical products produced by the chemical production network can be reliably attached to digital assets including the identifiers relating to chemical products and associated environmental attributes.

In another embodiment the identifier includes a chemical product identifier relating to the chemical product specification and one or more environmental attribute identifier(s) relating to one or more environmental attribute(s). In this embodiment the chemical product identifier and environmental attribute identifier are separated. The chemical product identifier may relate to the chemical product specification of a specific chemical product. The environmental attribute identifier may relate to one or more environmental attribute(s) assignable to the specific chemical product.

By using environmental attribute identifiers and chemical product identifiers separately, a flexible assignment of environmental attributes to the chemical product can be realized. By pre-defining the chemical product specification for the chemical product identifier and combining such chemical product identifier with the environmental attribute identifier, a reliable and flexible assignment of environmental attributes to the chemical product can be realized.

In another embodiment for assignment the environmental attribute identifier is linked to the chemical product identifier. In another embodiment the environmental attribute identifier relates to one environmental attribute or a combination of environmental attribute(s). The environmental attribute identifier may relate to one environmental attribute. This way the assignment of the environmental attribute may be done by linking one or more environmental attribute identifier(s) to the respective chemical product identifier. The environmental attribute identifier may relate to a set of environmental attribute(s). This way the assignment of the set of environmental attributes may be done by linking the environmental attribute identifier to the respective chemical product identifier.

In another embodiment for assignment one or more target environmental attribute(s) for the chemical product are provided and depending on the provided target environmental attribute(s) one or more environmental attribute identifier(s) are linked to the chemical product identifier. This way the environmental attributes assigned to a chemical product through linking of identifiers may be tailored to the needs of a customer buying the chemical product produced by the chemical production network.

In another embodiment for assignment the chemical product identifier is generated or selected based on the chemical product and/or the environmental attribute identifier is generated or selected based on the one or more environmental attribute(s) to be assigned from the balancing account or input material(s) to the chemical product. In another embodiment for assignment the chemical product identifier is generated based on the chemical product and/or the environmental attribute identifier is generated based on the one or more environmental attribute(s) to be assigned from the balancing account or input material(s) to the chemical product.

The chemical product identifier and the environmental attribute identifier may be unique identifiers for the chemical product, such as the specific chemical product, and the environmental attribute respectively. The chemical product identifier may be generated for chemical products, such as each chemical product, produced by the chemical production network. The chemical product identifier may be selected from a set of pre-generated chemical product identifiers for chemical products produced by the chemical production network. The chemical product identifier may be related to a physical identifier physically or virtually connected to the chemical product. The environmental attribute identifier may be generated for environmental attribute(s) available to the chemical production network. The environmental attribute identifier may be related to input materials entering the chemical production network, energy used by the chemical production network or processes of the chemical production network. The environmental attribute identifier may be selected from a set of pre-generated environmental attribute identifiers for environmental attributes available to the chemical production network.

In another embodiment the chemical product identifier is linked to a physical identifier attached to the chemical product and/or wherein the chemical product identifier is linked to a batch number and/or an order number associated with the chemical product. The chemical product identifier may be related to a physical identifier physically or virtually connected to the chemical product, such as the specific chemical product produced by the chemical production network. For physical linking a tag or code may be physically connected to the material, e.g. by printing an identifier number, QR code with embedded identifier or the like on the packaging. For virtual linking different identifiers associated with the physical material may be linked. For example, an order number, a batch number, a LOT number or a combination thereof may be linked together to uniquely identify the physical entity of the chemical product, such as the specific chemical product produced by the chemical production network.

### BRIEF DESCRIPTION OF THE DRAWINGS

FIG. 1 illustrates an example of a chemical production network producing one or more chemical product(s) from one or more input material(s) in connection with an operating system including an attribute management system.

FIGS. 2a-c illustrate examples of allocation schemes allocating the use of renewable or bio-based input materials to chemical products of the chemical production network.

FIG. 3 illustrates an example of a chemical production network with different allocation schemes.

FIGS. 4a,b illustrate inbound and outbound allocation of environmental attributes for a chemical production network.

FIG. 5 illustrates an example of a method for assigning at least one environmental attribute to at least one chemical product produced by a chemical production network.

FIG. 6 illustrates an example service environment for assigning at least one environmental attribute to at least one chemical product produced by the chemical production network.

FIG. 7 illustrates a flowchart of an example method for assigning at least one environmental attribute to at least one chemical product produced by the chemical production network.

FIG. 8 illustrates an example of chemical products produced by the chemical production network.

FIG. 9 illustrates an example of the converting to balancing units and assignment to chemical products.

FIGS. 10a,b illustrate examples of data structures for assigning environmental attributes from the balancing account to the chemical product identifier.

FIG. 11 illustrates an example of a method for producing at least one chemical product associated with at least one environmental attribute for a supply chain.

### DETAILED DESCRIPTION

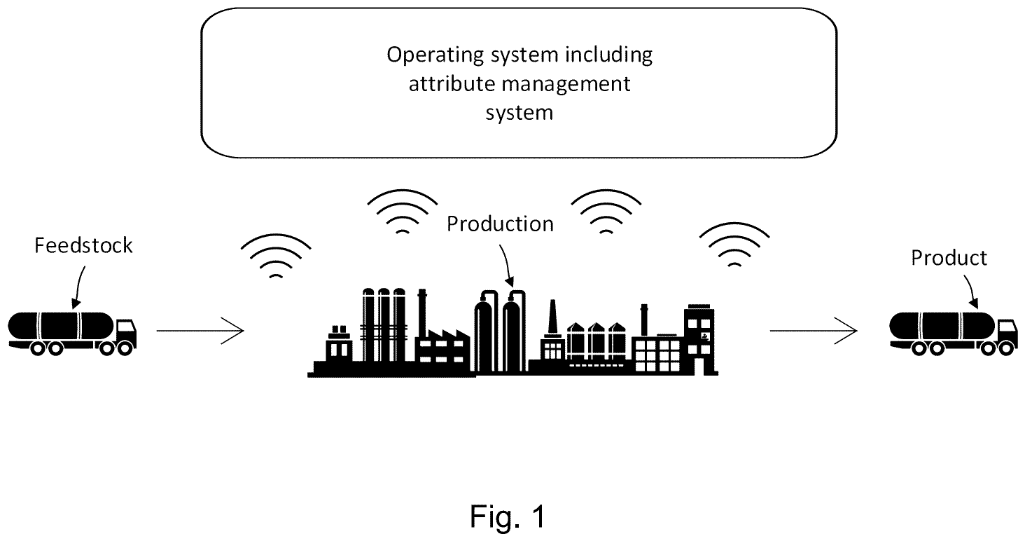

FIG. 1 illustrates an example of a chemical production network producing one or more chemical product(s) from one or more input material(s) in connection with an operating system including an attribute management system.

For producing one or more chemical product(s) different input materials may be provided as physical inputs from material providers or suppliers. The input materials or the chemical products produced from the input materials may have one or more properties related to the environmental impact of the input materials or the chemical products produced from the input materials.

The chemical production network may include multiple interlinked processing steps. The chemical production network may be an integrated chemical production network with connected or interconnected production chains. The chemical production network may include multiple different production chains that have at least one intermediate product in common. The chemical production network may include multiple stages of the chemical value chain. The chemical production network may include the producing, refining, processing and/or purification of gas or crude oil. The chemical production network may include a stream cracker, or a syngas plant connected to multiple production chains that output chemical products from the effluent of the steam cracker or syngas plants. The chemical production network may include multiple production chains that produce from one or more input material(s) chemical products that exit the chemical production network. The chemical production network may include multiple tiers of a chemical value chain. The chemical production network may include physically connected or interconnected production sites. The production sites may be at the same location or at different locations. In the latter case, the production sites may be connected or interconnected by means of dedicated transportation systems such as pipelines, supply chain vehicles, like trucks, supply chain ships or other cargo transportation means.

The chemical production network may chemically convert input materials via chemical intermediates to one or more chemical products that exit the chemical production network. The chemical production network may convert input materials by way of chemical conversion to one or more chemical products.

The input materials may be fed into the chemical production network at any entry point. The input materials may be fed into the chemical production network at the start of the chemical production network. Input materials may for example make up the feedstock of a steam cracker. The input material may include a bio-based, a recycled and/or a fossil input material for the manufacture of chemical intermediates and chemical products.

The chemical production network may include multiple production steps. The production steps included in the chemical network may be defined by the system boundary of the chemical production network. The system boundary may be defined by location or control over production processes. The system boundary may be defined by the site of the chemical production network. The system boundary may be defined by production processes controlled by one entity or multiple entities jointly. The system boundary may be defined by value chain with staggered production processes to an end product, which may be controlled by multiple entities separately. The chemical production network may include a waste collection and sorting step, a recycling step such as pyrolysis, a cracking step such as steam cracking, a separation step to separate outputs of one process step and further processing steps to convert such outputs to a chemical product leaving the system boundary of the chemical production network.

The operating system of the chemical production network may monitor and/or control the chemical production network based on operating parameters of the different processes. One process step monitored and/or controlled may be the feed of input materials or the release of chemical products. Another process step monitored and/or controlled may the allocation of environmental attributes to chemical products produced via the chemical production network. Yet another process step monitored and/or controlled may be the registration of environmental attributes associated with input materials entering the system boundary of the chemical production network. Yet another process step monitored and/or controlled may be the management of environmental attributes associated with input materials and chemical products of the chemical production network.

The operating system may be configured to access data related the inputs materials, the processes and/or the chemical products produced by the chemical production network. The operating system may be configured to convert a recycled, renewable or bio-based content of the one or more input material(s) used in the chemical production network to balancing units. The operating system may be configured to allocate the balancing units to at least one balancing account associated with the recycled or bio-based content of the input materials. The operating system may be configured to allocate at least a part of the balancing units from the at least one balancing account to the at least one chemical product.

The operating system may be configured to handle balancing units related to the input and chemical products produced by the chemical production network. In particular, the operating system may be configured to determine balancing units associated with the use of input materials impacting the environmental property of the chemical production network and the chemical products produced by the chemical production network. Further in particular, the operating system may be configured to determine balancing units associated with the chemical products and the environmental property of the chemical products. This way the operating system may be configured to allocate balancing units to balancing accounts or to deallocate balancing units from the balancing accounts. The balancing units may be viewed as a credit that may be deposited in an account or deducted from an account related to the input and chemical products of the chemical production network.

The operating system may be configured to register inbound environmental attributes, to assign outbound environmental attributes and to manage inbound allocation as well as outbound assignment.

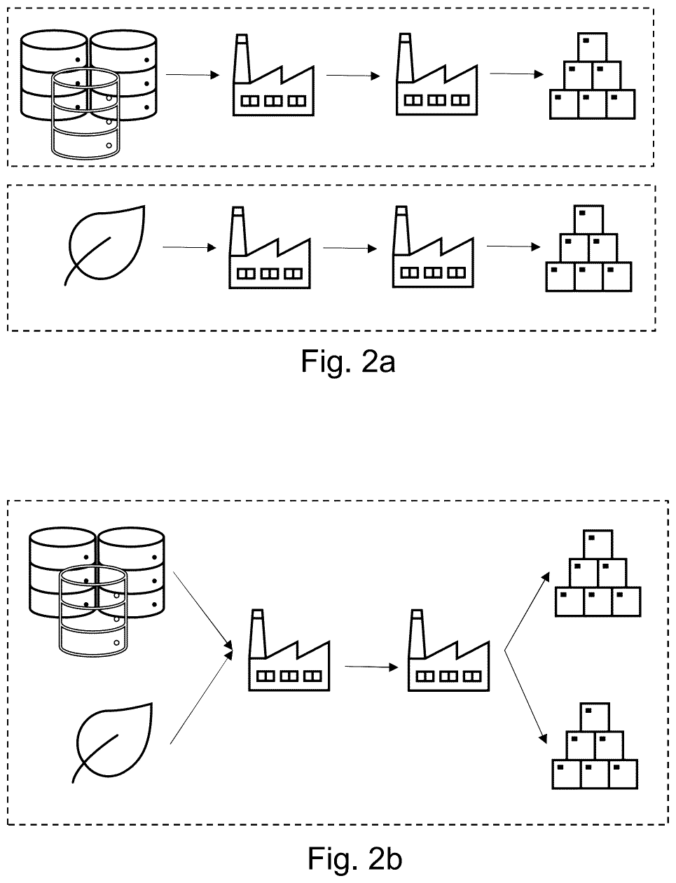

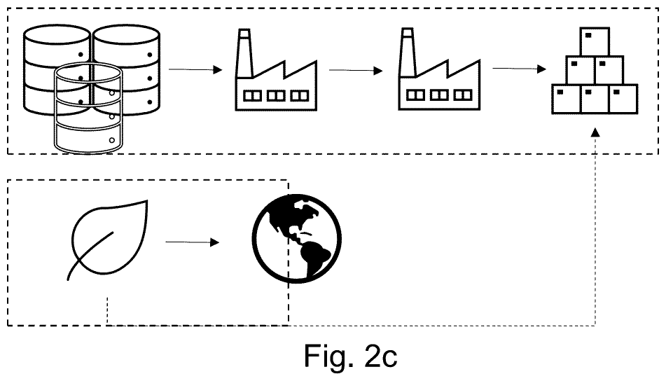

FIGS. 2a to c illustrate examples of allocation schemes allocating the use of renewable or bio-based input materials to chemical products of the chemical production network.

As illustrated in FIG. 1 chemical production networks can comprise complex interconnected production sites that chemically convert one or more input materials via chemical processing to one or more chemical products. To account for the use of recycled, bio-based or renewable content in chemical production, allocation rules may be used. This way recycled, renewable or bio-based content of input materials may be allocated to chemical products. The renewable content may be based on input material from renewable sources. The renewable content may comprise bio-based input materials produced from living organisms such as different types of crops, wood or algae. The recycled content may comprise any recycled material used in production of new materials. This may include any recycled bio-based or bio-based materials e.g. as produced from chemical or mechanical recycling.

Accounting principles for allocating the use of recycled or renewable content are for example defined in ISO 22095. Four different models may be used: Identity preservation models, segregated models, mass balance models, or book and claim models.

FIG. 2a illustrates an example of a dedicated or segregated production network. The production network comprises a first production chain for producing the chemical product from fossil material and a second production chain for producing the chemical product from bio-based input materials. The first and the second production chain are not interconnected. The first and the second production chain produce a fossil-based and a bio-based output material, respectively. Example for such dedicated production environments include fermentation or chemical transformation, such as polyethylene production from sugar cane, bio-poly lactic acid (PLA) production from corn, bio-succinic acid, or bio-butanediol (BOO).

FIG. 2b illustrates an example of a complex production network. In contrast to the production network of FIG. 2a the fossil-based input materials are co-fed and mixed with bio-based input materials. The production network produces via one or more chemical process chains with intermediates one or more material outputs or products. For the sake of simplicity FIG. 2b illustrates the mass balancing approach for one chemical process chain producing one chemical product or product. In the mass balancing model, the physical mixing or co-feeding of bio-based input material with conventional fossil input materials is accounted for. Here the feed into the production network and the feed of output products form a system boundary. The mass balance of input and output products connect the used bio-based input material to the produced chemical product. Mass balance allows to keep track of the total amount of input material (e. g. recycled or bio-based or bio-based materials) throughout the production network and allows for allocation to chemical products. Materials with different sets of specified characteristics may be mixed. E. g. recycled or bio-based feedstock replaces an equivalent amount of fossil feedstock at the beginning of the value chain (input material) and is allocated to a product (output material) in such a manner that the input and output match. For this model, the proportion of the input with specified characteristics might only match the initial proportions on average and will typically vary across different outputs. This means that e.g. renewable and fossil input materials are mixed and that the chemical or technical proportions in each chemical product are not tracked.

Mass balance may include conversion factors to ensure the amount of input material is correlated with the amount of output material. The calculation may be made over a pre-defined or specified time period. Mass balance may be based on a balancing unit such as mass, energy or carbon.

Like FIG. 2b, FIG. 2c illustrates a complex production network associated with a book and claim scheme. In book and claim scheme, the characteristic renewable or recycled input material is not linked to the actual material flows. Book & Claim allows to de-couple specific characteristic, such as renewable, from the physical product and to transfer the characteristic separately via a dedicated registry in the form of a digital asset. This approach may be used for renewable energy. Book and claim may be based on a book and claim accounting unit such as watts or kilowatts for electricity.

In identify preservation models or segregated approaches as illustrated in FIG. 2a renewable, recycled or bio-based input materials may not be mixed with fossil input material. In mass balance or book and claim approaches as illustrated in FIGS. 2b and 2c renewable or recycled or bio-based materials with fossil input material may be mixed. In view of the increasing number of different sources for more sustainable chemical production and the number of allocation schemes, an efficient and robust operation system for operating complex production networks like chemical production networks is required.

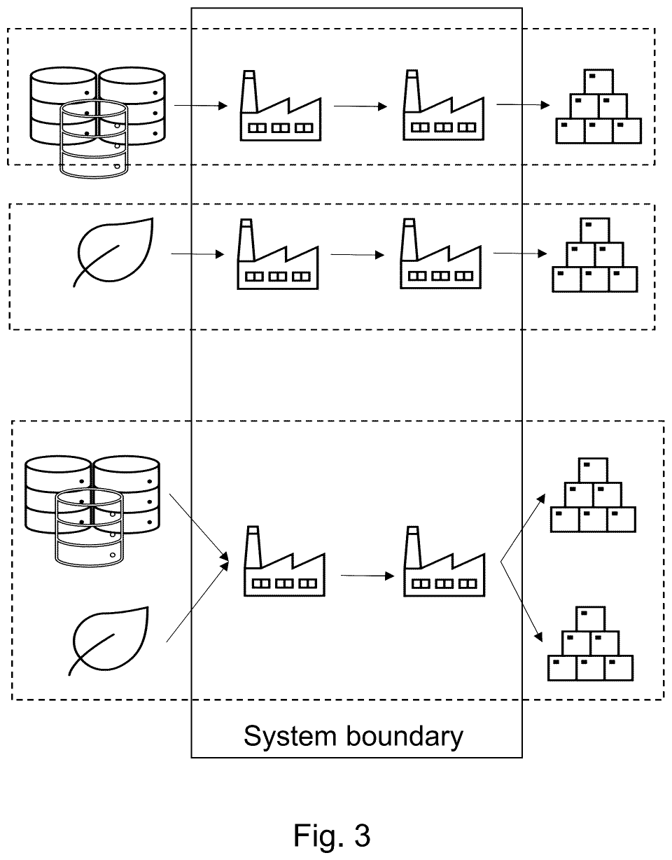

FIG. 3 illustrates an example of a chemical production network with different allocation schemes.

The chemical production network may include multiple production chains with different allocation schemes. The system boundary of the chemical production network may signify the entry points to the chemical production network and the exit points from the chemical production network. The production chains may be signified by the chemical product(s) produced via such production chains. The production chain logic may be based on process data associated with process steps from input material(s) to chemical product(s). For each production chain an allocation scheme may be applicable or assign to.

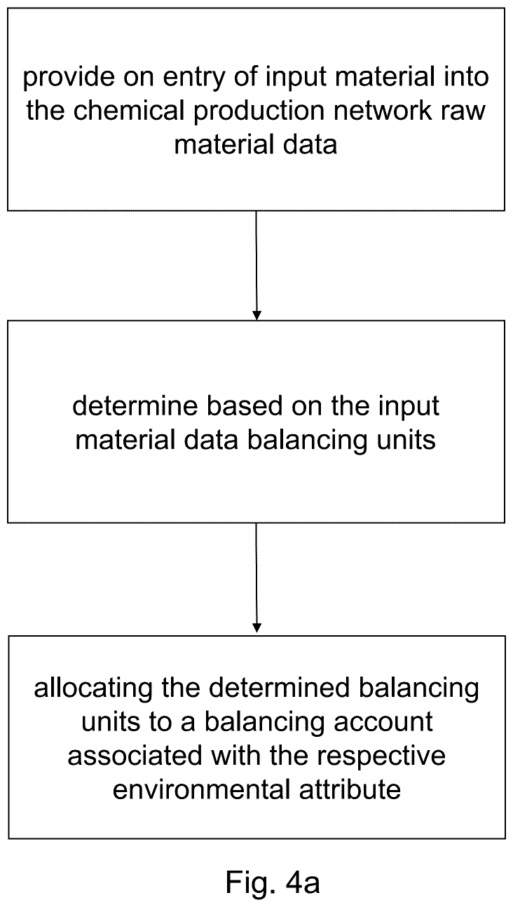

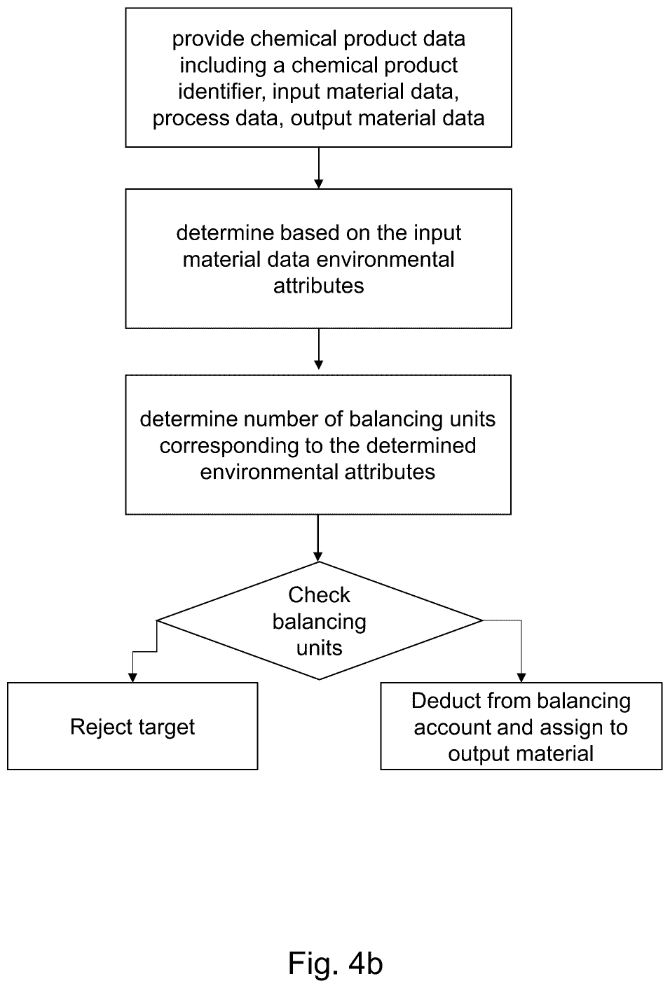

FIGS. 4a,b illustrate inbound and outbound allocation of environmental attributes for a chemical production network.

FIG. 4a illustrates an example of an inbound allocation process for the chemical production network.

As for example illustrated in FIG. 1 the chemical production network may be operated by the operating system configured to register inbound environmental attributes, to assign outbound environmental attributes and/or to manage inbound registration as well as outbound assignment via allocation rules. The allocation rules may include allocation schemes as illustrated in FIGS. 2a-c. The chemical production network may include the network as illustrated in FIG. 3.

On input material(s) entering the chemical production network or the physical system boundary of the chemical production network the inbound allocation takes place. In this context input material signifies any material that enters the system boundary of the chemical production network and is used to produce chemical products leaving the chemical production network. Such input material(s) may be associated with an input material identifier. Possible identifiers include decentral identifiers, input material specification, input material order number, input material LOT number, input material batch number, input material supplier specification or combination thereof.

On entry of the input material into the chemical production network input material data may be registered. The input material data may include input material identifier, input material amount and/or one or more environmental attribute(s) associated with the input material.

Based on the input material data, in particular the input material amount and/or one or more environmental attribute(s) associated with the input material, balancing units may be determined by converting the input material amount to balancing units for the respective environmental attribute. The balancing units may relate to carbon atoms, methane equivalents, or any other suitable measure for the environmental impact of the environmental attribute. For example, the conversion to balancing units may include the conversion of the input material amount to an energy property including material losses that occur during production of output product(s).

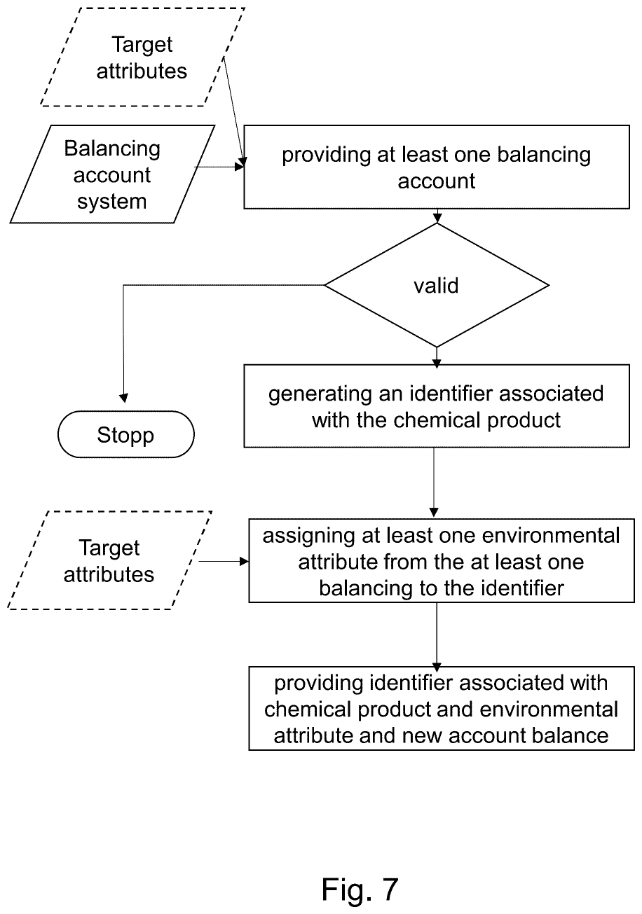

The balancing units may be allocated to a balancing account associated with the respective environmental attribute. This way the environmental attributes associated with input material(s) entering the chemical production network can be tracked or stored in an accounting system. An example of an accounting system is shown in FIGS. 7a-d, where each balancing account is associated with metadata signifying the input material such as pyrolysis oil, environmental attribute type such as recycled material and material origin such as recycled from tires. For allocation the input material data may include respective metadata for the input material. Allocation rules may include instructions to allocate the environmental attribute(s) included in the input material data to the respective balancing account. In the inbound process such allocation rules may be used to allocate environmental attributes from the input material data to the balancing system.

For example, the method for allocating balancing units may be associated with pyrolysis oil as input material to a complex chemical production network to the output products of the complex chemical production network.

In a first step the quantity and type of waste is provided to a computing interface. The quantity and type of waste are stored in connection with the pyrolysis plant(s) and pyrolysis oil produced by plant. For instance, a pyrolysis oil producer may operate multiple plants and may track the quantity and type of waste fed to the plants. The waste may be provided separately according to type of waste or the waste may be mixed. Based on such tracking the quantity of pyrolysis oil and type of waste may be allocated to the produced pyrolysis oil.

In a second step the amount of pyrolysis oil and the type of waste may be provided to a second computing system associated with the operating system of a complex chemical production network. Based on the quantity of pyrolysis oil balancing units may be generated. Such generation may include a conversion factor that takes account of the chemical difference between fossil-based input materials such as naphtha and methane. In one example the conversion factor may relate to the lower heating value of the pyrolysis oil in relation the lower heating value of naphtha or methane. The conversion factor may for instance include the ratio of the lower heating value of pyrolysis oil and naphtha or methane. This way the chemical difference between the fossil and the recycled input material can be taken into account.

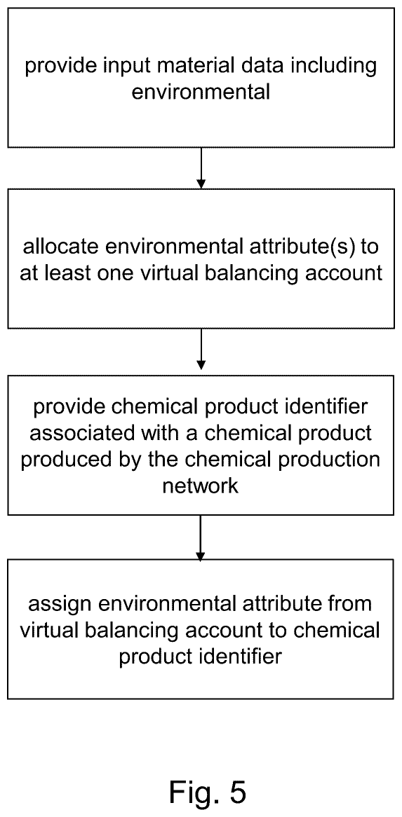

The balancing units may be allocated to the respective balancing account. For example the allocation rule may be associated with balancing account 1 of FIG. 5. In such a case the balancing units for the pyrolysis oil produced from waste of the waste type and used in a specified quantity in the production network will be allocated to balancing account 1. On production of the output products such balancing units may be allocated to the chemical product according to the allocation rule. An allocation rule that specifies free attribution between production processes of the chemical production network provides for reliable allocation in complex production networks.

FIG. 4b illustrates an example of an outbound allocation process for the chemical production network.

As for example illustrated in FIG. 1 the chemical production network may be operated by the operating system configured to register inbound environmental attributes, to assign outbound environmental attributes and/or to manage inbound registration as well as outbound assignment via allocation rules. The allocation rules may include allocation schemes as illustrated in FIGS. 3a-c. The chemical production network may include the network as illustrated in FIG. 2.

The chemical product may be produced from input material that entered the chemical production network. Chemical product data including an chemical product identifier may be provided. The chemical product data may further include input material data associated with the input materials used to produce the chemical product, process data associated with the production chain for producing the chemical products and/or chemical product data associated with the chemical products, such as an chemical product specification or an chemical product amount.

Based on the input material data environmental attributes associated with the input material(s) used to produce the chemical product may be determined. The input material data may specify the total amount of input material(s) that entered the production chain for producing the amount of chemical product. The input material data may further specify the environmental attributes available for the respective input material(s). From the total amount of input material(s), the number of balancing units for respective environmental attributes available for such input material(s) may be determined. This way the maximum number of balancing units for the respective environmental attribute attributable to the chemical product may be determined.

Based on the environmental attribute(s) attributable to the chemical product, the number of balancing units corresponding to the determined environmental attributes may be determined. The determined balancing units may be compared to balancing units stored in balancing account(s) for the respective environmental attribute(s).

If the balance of the respective balancing account(s) for the respective environmental attribute(s) is not sufficient, the environmental attribute(s) is rejected. If the balancing units are available, the balancing units are deducted from the respective balancing account(s) and the environmental attribute(s) are assigned to the chemical product and

Each balancing account may be associated with metadata signifying the input material such as pyrolysis oil, environmental attribute type such as recycled material and material origin such as recycled from tires. For assignment of the environmental attribute(s) to the chemical product, allocation rules may be defined. Allocation rules may include instructions to assign the environmental attribute(s) included in the input material data from the respective balancing account to the chemical product(s). In the outbound process such allocation rules may be used to assign environmental attributes from the balancing system to the chemical product(s).

FIG. 5 illustrates an example of a method for assigning at least one environmental attribute to the least one chemical product produced by a chemical production network.

The one or more input material(s) may be provided at entry points to the chemical production network. Material data related to the one or more input material(s) and respective environmental attributes may be provided to a computing interface. The material data may be provided on, prior or after entry of the one or more input material(s) to the chemical production network. The material data may include the input material identifier associated with the respective input materials provided to the chemical production network. The input material identifier may be associated with the physical entity of the input material. This way the virtual identifier of the input material may be uniquely linked to the physical input material. Such linking may include a physical or virtual link of identifiers uniquely associated with the physical input material. For physical linking a tag or code may be physically connected to the material, e.g. by printing a QR code on the packaging. For virtual linking different identifiers associated with the physical material may be linked. For example, an order number, a batch number, LOT number or a combination thereof may be linked. The material data may include at least one environmental attribute associated with the respective input material and an amount of input material provided to the entry point of the chemical production network.

The one or more environmental attribute(s) may be allocated to at least one virtual balancing account associated with the respective environmental attribute. The virtual balancing account may be associated with a digital storage structure that stores data related to environmental attributes. The account may be associated with meta data identifying the account for balancing environmental attributes. The account may be associated with meta data identifying the environmental attributes and the environmental units allocated to the account. The account may be associated with meta data identifying the production chain the account is associated with. The account may be associated with meta data identifying the input or chemical product the account is associated with. The account may be part of a balancing system including multiple accounts. The account may hold environmental attributes for transaction. Environmental attributes may be allocated, added, deleted, withdrawn or deducted from the account. The virtual balancing account may by associated with environmental attribute types such as recycled or renewable. The virtual balancing account may by associated with input material types such as pyrolysis oil, bio-naphta, bio-methane, or combinations thereof. The virtual balancing account associated with the environmental attribute type recycled may be further associated with waste-stream type such as mixed plastics waste, specific end product waste, e.g. tiers waste or foam waste, post-consumer waste, pre-consumer waste or combinations thereof. The virtual balancing account may by associated with an allocation scheme such as segregated allocation, non-segregated allocation such as book and claim, mass balance with free attribution, mass balance without free attribution or combinations thereof.

The chemical product identifier associated with a chemical product produced by the chemical production network and provided at the exit point from the chemical production network may be provided. The chemical product identifier may be associated with the physical entity of the chemical product. This way the virtual identifier of a material may be uniquely linked to the physical material. Such linking may include a physical or virtual link of identifiers uniquely associated with the physical material. For physical linking a tag or code may be physically connected to the material, e.g. by printing a QR code on the packaging. For virtual linking different identifiers associated with the physical material may be linked. For example, an order number, a batch number, LOT number or a combination thereof may be linked.

At least one environmental attribute from the at least one virtual balancing account associated with the respective environmental attribute may be assigned to the at least one chemical product identifier. This way the chemical product may be provided together with the digital asset of the environmental attribute and the positive environmental impact associated with the chemical product can be tracked through the value chain using such chemical product. The tracking may even stretch to the end product produced from the chemical product.

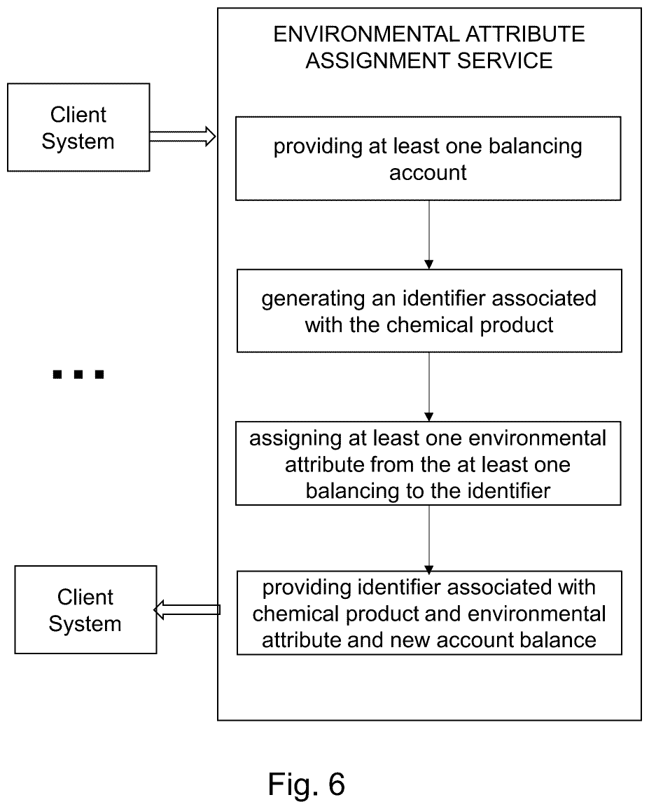

FIG. 6 illustrates an example service environment for assigning at least one environmental attribute to at least one chemical product produced by the chemical production network.

The example service environment of FIG. 6 includes a number of client systems, represented by two example client systems. The client systems can be operated by the same organization or by different organizations. The client systems are involved with the assignment of environmental attributes to at least on chemical product. For example, one client system could be a system (e.g., comprising server resources, database resources, storage resources, software resources, etc.) of an organization that is involved with the receiving of materials. In some implementations, the client systems are ERP systems.

The example environment also includes an environmental attribute assignment service. The environmental attribute assignment service may be implemented by a variety of server resources, database resources, storage resources, software resources, and/or other types of computing resources. The environmental attribute assignment service provides assignment services to the client systems.

An assignment process may be initiated when one of the client systems sends a request to assign environmental attributes from balancing account system to at least one chemical product produced by a chemical production network. The request may include balancing accounts associated with one or more environmental attribute(s) and the account balance associated with one or more environmental attribute(s) from one or more input material(s) provided to the chemical production network. The request may further include target attributes signifying the environmental attributes to be assigned to the chemical product. Based on the environmental attribute, the target attribute and the balance the validity of the request may be checked, for instance it the provided balancing account(s) hold sufficient balance.

The assignment service, upon valid request may provide or generate the identifier associated with the chemical product. The identifier may be a chemical product identifier relating to a chemical product specification including one or more environmental attribute(s). The chemical product identifier may relate to a pre-defined chemical product specification and one or more pre-defined environmental attribute(s). The chemical product identifier may relate to a pre-defined chemical product specification and one or more pre-defined set(s) of one or more environmental attribute(s). Alternatively or additionally, the identifier may include a chemical product identifier relating to the chemical product specification and one or more environmental attribute identifier(s) relating to one or more environmental attribute(s). The environmental attribute identifier may relate to one environmental attribute or a combination of environmental attribute(s). The chemical product identifier may be generated based on the chemical product and/or the environmental attribute identifier may be generated based on the one or more environmental attribute(s) to be assigned from the balancing account or input material(s) to the chemical product.

Upon generation of the identifier, at least one environmental attribute from the at least one balancing account associated with the respective environmental attribute may be assigned to the at least one chemical product. The environmental attribute from the balancing account may in particular be assigned to the generated or provided identifier. Depending on the identifier structure, the environmental attribute identifier may be linked to the chemical product identifier.

For assignment one or more target environmental attribute(s) for the chemical product may be provided and depending on the provided target environmental attribute(s) one or more environmental attribute identifier(s) may be linked to the chemical product identifier. The chemical product identifier may be generated or selected based on the chemical product and/or the environmental attribute identifier may be generated or selected based on the one or more environmental attribute(s) to be assigned from the balancing account or input material(s) to the chemical product. The chemical product identifier may be linked to a physical identifier attached to the chemical product. The chemical product identifier may be linked to a batch number and/or an order number associated with the chemical product. The identifier associated with chemical product and environmental attribute and/or the new account balance may be provided.

This way the at least one environmental attribute from the at least one balancing account associated with the respective environmental attribute may be assigned to the at least one chemical product. The method lined out here may be used in the context of the chemical production network for producing at least one chemical product associated with one or more environmental attributes. One or more input material(s) associated with one or more environmental attribute(s) to the chemical production network. The at least one chemical product may be produced by the chemical production network. Data related to the input materials associated with one or more environmental attributes and the production of chemical products may be provided to the operating system as described in the context of FIG. 1. The operating system may be configured to perform the assignment method described herein.

FIG. 7 illustrates a flowchart of an example method for assigning at least one environmental attribute to at least one chemical product produced by the chemical production network.

For assigning at least one environmental attribute to at least one chemical product produced by the chemical production network providing at least one balancing account associated with environmental attributes and the environmental attribute balance may be provided. In addition, the target attributes may be provided. If e.g. the account is valid for the target attributes and/or the balance is sufficient for assignment, the identifier associated with the chemical product is generated. Based on the generated identifier the at least one environmental attribute may be assigned from the at least one balancing account to the identifier. The identifier associated with chemical product and environmental attribute and/or the new account balance may be provided.

Further details of the method are described in the context of FIG. 6 and the following figures.

For illustration purposes and to further explain the methods shown in FIGS. 1-7, FIG. 8 shows an example of chemical products produced by the chemical production network from input materials through different intermediates.

The chemical production network may include multiple production chains with one or more intermediate stage. The production chains may have one common starting point as for example provided by the steam cracker. The cracker feedstock may comprise fossil and non-fossil input materials. The fossil input materials may comprise gaseous or liquid hydrocarbon containing feedstock such as naphtha and/or low-pressure gas or ethane. The non-fossil input materials may comprise bio-based and/or recycled feedstock.

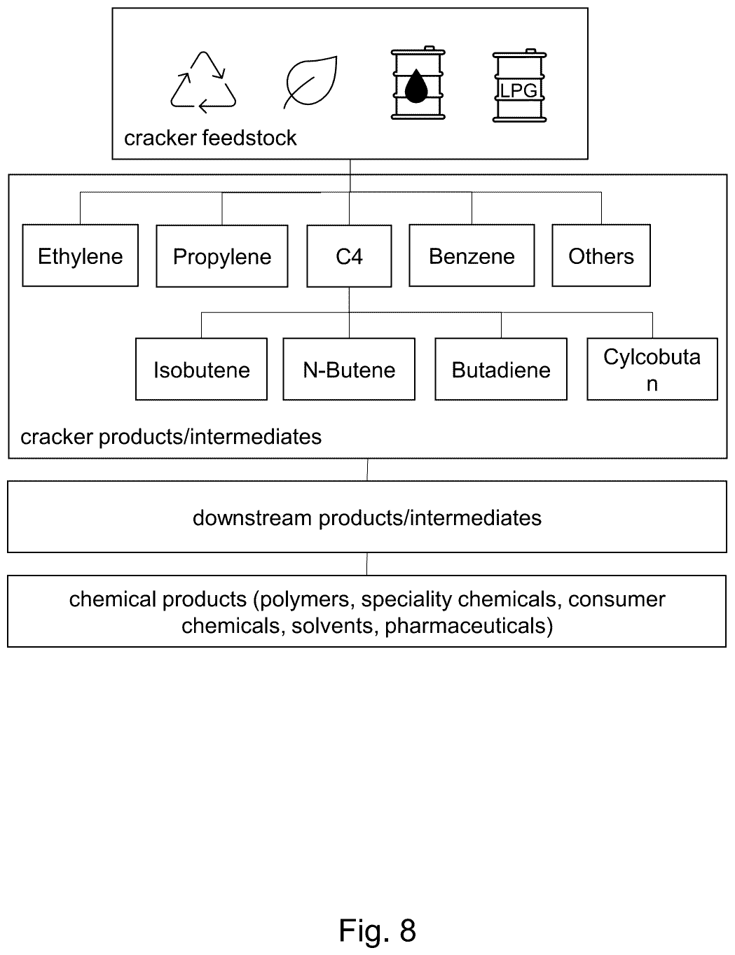

In steam cracking the feedstock is diluted with steam and heated in a furnace to reaction temperatures between 500-1000° C., for example around 850° C. After the cracking, the gas is quenched to stop the reaction in a transfer line exchanger. The cracker products produced in the reaction depend on the composition of the feed, on the hydrocarbon to steam ratio and on the cracking temperature and furnace residence time. In the example shown in FIG. 8, cracker products include light alkenes such as ethylene or propylene, C4-hydrocarbons such as Isobutene, N-Butene, Butadiene or Cyclobutane, and/or other hydrocarbons such as aromatic hydrocarbons or mixtures of aromatic hydrocarbons, such as benzene, toluene and/or xylenes (benzene and toluene also abbreviated as BT; benzene, toluene and xylenes also abbreviated as BTX).

The cracker products may be provided to the exit point of the chemical production network. The cracker products may in such embodiment be chemical products. The environmental attributes of the recycled or bio-based input materials may be assigned to such chemical products.

The chemical production network may include one or more production chain(s) that further process or chemically convert the cracker intermediates. The cracker intermediates may be further processed or chemically converted by one or more production chain(s) inside the chemical production network. The cracker intermediates may be processed to downstream products. The downstream products may be provided to the exit point of the chemical production network. The downstream products may hence be chemical products. The environmental attributes of the recycled or bio-based input materials may be assigned to such chemical products. The cracker intermediates may be processed via one or more downstream intermediates to chemical products. The environmental attributes of the recycled or bio-based input materials may be assigned to such chemical products. Chemical products may include polymers, specialty chemicals, consumer chemicals, solvents, pharmaceuticals or the like.

For illustration purposes and to further explain the methods for allocating environmental attributes to virtual balancing accounts and assigning environmental attributes to chemical products as described in the context of FIGS. 1-8, FIG. 9 illustrates examples of the conversion to balancing units and assignment to chemical products.

Based on the non-limiting example of FIG. 8 bio-based and recycled feedstock may be provided to the steam cracker. For illustrative purposes the following input materials may be provided to the steam cracker in the following amounts:
3 kg recycled input material such as pyrolysis oil produced from mixed plastics waste,
5 kg bio-based input material such as bio-naphtha from vegetable oil that is kosher and vegan,
92 kg of naphtha.

On providing these input materials to the steam cracker, the input materials enter the chemical production network. The environmental attributes of the recycled and bio-based input materials may be provided to the computer interface configured to convert such attributes to balancing units and to allocate the balancing units to respective virtual balancing accounts. In the simplest example, the balancing units (BUs) may correspond to the amounts of the respective materials. In the example, the environmental attribute pyrolysis oil from recycled mixed plastics waste may correspond to 3 BUs and the environmental attribute bio-naphtha from bio-based food waste may correspond to 5 BUs.

In more elaborate embodiments the balancing units may be determined based on a more complex conversion factor taking chemical and/or physical differences between input materials and their associated yield into account. The conversion factor may quantify the differences in chemical and/or physical properties of replacing fossil input material(s) by non-fossil input material(s). The conversion factor may relate the use of conventional input material(s) to the use of input material(s) associated with one or more environmental attribute(s). The conversion factor may depend on carbon atoms, methane molecules, energy properties, process properties or any other suitable factors for quantifying the environmental impact of the environmental attribute. For instance, the lower or higher heating value (LHV, HHV) of the fossil and the non-fossil input material may be taken into account. Further for instance, material losses that occur in the processing of the fossil or the non-fossil input material may be taken into account. Further for instance, exempted steam cracker products, intermediates or production chains may be taken into account. Further for instance, only pre-selected production chains may be taken into account. This way the environmental impact of the non-fossil input materials may be quantified with reference to fossil input materials.

The steam cracker may produce cracker products, which may be further processed and chemically converted. In the illustrative example, 20 kg ethylene as cracker product, 30 kg polyamide and 50 kg polystyrene may be provided to the exit point of the chemical production network. Since for the production of such products 3 kg recycled and 5 kg bio-based input materials were used, the environmental attributes allocated in BUs to the respective balancing accounts can be assigned to such products. For instance, 3 BUs recycled may be assigned to Polyamide, which corresponds to 10% recycled content, and 5 BUs bio-based may be assigned to Polystyrene, which corresponds to 10% bio-based content.

For illustration purposes and to further explain the methods for assigning environmental attributes to chemical products as described in the context of FIGS. 1-9, FIG. 9 illustrates examples of virtual balancing accounts to manage the allocation and assignment of balancing units.

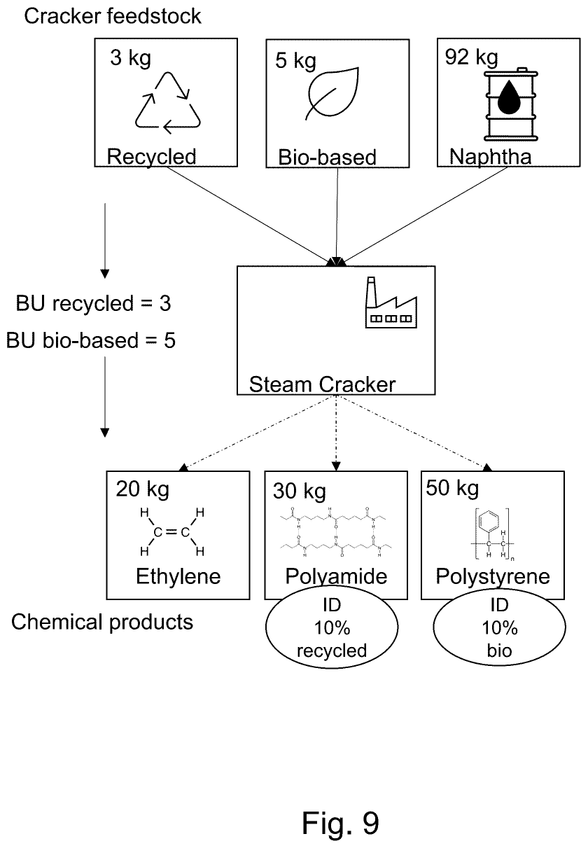

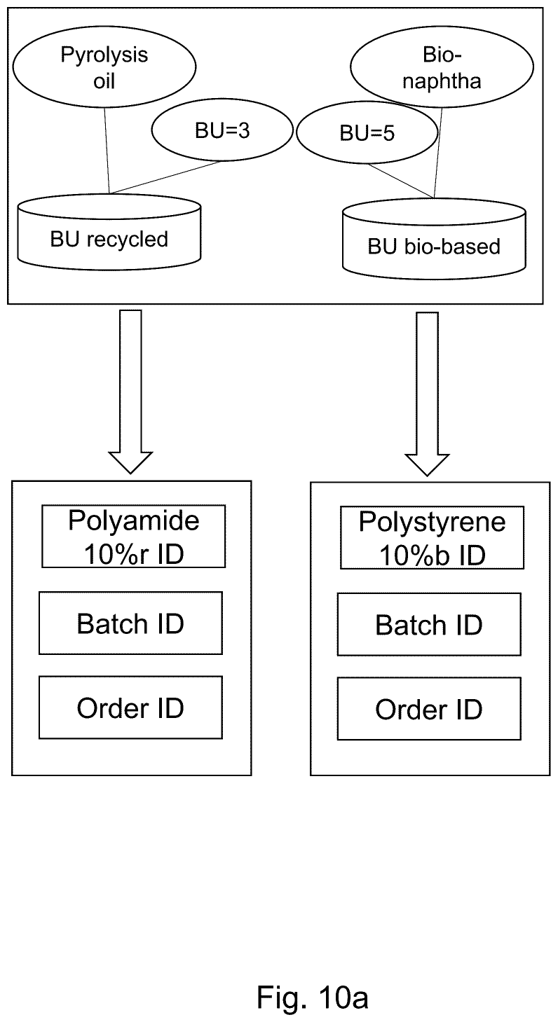

Based on the non-limiting example of FIG. 9 bio-based and recycled feedstock may be provided to the steam cracker. Material data related to the bio-based and recycled feedstock and the respective environmental attributes may be provided to the computing interface configured to convert such attributes to balancing units and to allocate the balancing units to respective virtual balancing accounts. In the simplest example, the environmental attribute pyrolysis oil from recycled mixed plastics waste may correspond to 3 BUs and the environmental attribute bio-naphtha from bio-based vegetable oil may correspond to 5 BUs. The so determined BUs may further be associated with metadata signifying the respective environmental attributes as recycled, pyrolysis oil and recycled tyers as well as bio-based, bio-naphtha, vegetable oil.

For allocation the virtual balancing system may be provided. The virtual balancing system may include balancing accounts associated with metadata relating to environmental attribute type, chemical production network, production chain of the chemical production network, allocation scheme, input material type, input material origin, chemical product type or combinations thereof.

The balancing accounts may be associated with meta data relating to environmental attribute type recycled or bio-based, input material type pyrolysis oil, bio-naphtha or bio-gas, input material origins tyers, mixed plastics waste, vegetable oil or food waste, respectively. Furthermore, the balancing accounts may be associated with an account balancing quantifying the BUs.

For allocation of the 3 BUs and the 5 BUs the metadata of the balancing accounts may be matched with the metadata associated with such BUs. Once a match in metadata is found the respective BUs are allocated to the matched account. Hence in the example, the 3 BUs pyrolysis oil from recycled mixed plastics waste may be allocated to account BU recycled 2 and the 5 BUs bio-naphtha from bio-based vegetable oil may be allocated to account BU bio 1. The virtual balancing system may be based on balancing accounts with static or dynamic meta data. For instance, if metadata provided via the environmental attribute associated with the input material does not correspond to any metadata of balancing accounts provided, a new account associated with such metadata may be provided. Alternatively or additionally, a hierarchical tree of balancing accounts (leafs) with different levels of metadata associated to the balancing accounts may be provided. For example the top level may be bio-based or recycled, the next level may specify the input material type for recycled or bio-based, respectively, and so on. Alternatively or additionally, the environmental attribute may be allocated to the account associated with the greatest match in metadata. Here greatest may refer to the maximal number of matching meta data points, in particular account metadata points matching at least in part with environmental attribute metadata points. For example, the environmental attribute may provide more metadata than any balancing account. In such a scenario the account with metadata points matching at least in part with metadata points of the environmental attribute may be selected for allocation.

For illustration purposes and to further explain the methods for assigning environmental attributes from the balancing account to the chemical product identifier as described in the context of FIGS. 1-9, FIG. 10a,b illustrate examples of data structures for assigning environmental attributes from the balancing account to the chemical product identifier.

Based on the non-limiting example of FIG. 8 bio-based and recycled feedstock may be provided to the steam cracker, converted to the balancing units of as shown in FIG. 9 and allocated to the balancing accounts. Balancing account recycled has in this example a balance of 3 BUs and balancing account bio-based has a balance of 5 BUs.

For assignment of such BUs to the chemical product, the chemical product identifier may be provided. The chemical product identifier may be associated with the chemical product provided to the exit point of the chemical production network. The chemical identifier may relate to the chemical product specification. The chemical identifier may relate to the chemical product specification and the environmental attribute.

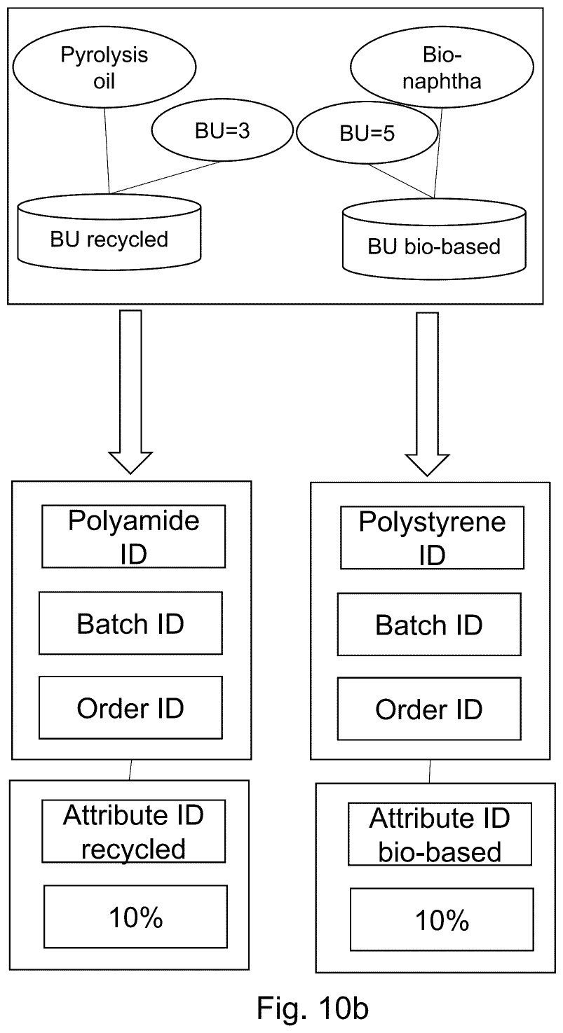

As shown in FIG. 10a, the chemical identifier may relate to the chemical product specification Polyamide or Polystyrene and the environmental attribute 10% recycled or bio-based content. The chemical product identifier may be provided for pre-defined chemical products associated with pre-defined environmental attribute(s). In this embodiment, the number of BUs required for the respective chemical products is pre-defined and a further conversion of BUs to respective environmental attribute(s) is not required. This way the management of input materials and chemical products with environmental attributes is less dynamic and can be simplified.

As shown in FIG. 10b, in addition to the chemical identifier an environmental attribute identifier may be provided. The chemical identifier may relate to the chemical product specification Polyamide or Polystyrene. The chemical product identifier may be provided for pre-defined chemical products. The environmental attribute identifier may relate to the environmental attribute 10% recycled or bio-based content. The environmental attribute identifier may be linked to the chemical product identifier. The environmental attribute identifier may be provided for pre-defined environmental attribute types. For assignment, the BUs may be assigned to the environmental attribute identifier. For assignment, the BUs may be converted to the environmental attribute and the environmental attribute such as 10% recycled content may be assigned to the environmental attribute identifier. In this embodiment, the number of BUs required for the respective chemical products is not pre-defined and can be flexibly assigned. This way chemical products with environmental attributes tailored to customer needs can be provided.

The chemical identifier may be uniquely linked to the physical entity of the chemical product. In one embodiment, the batch identifier and the order identifier may be provided and/or linked to chemical product identifier. This way the chemical product identifier may be uniquely linked to the physical entity of the chemical product exiting the chemical production network. In other embodiments the chemical identifier may be linked to the physical entity of the chemical product by way of a physical identifier with encoded chemical product identifier and physically connected to the chemical product. For example, a tag or a QR code may be physically connected to the chemical product and the chemical product identifier may be encoded into the tag or QR code. This way the chemical product identifier may be uniquely linked to the physical entity of the chemical product exiting the chemical production network.

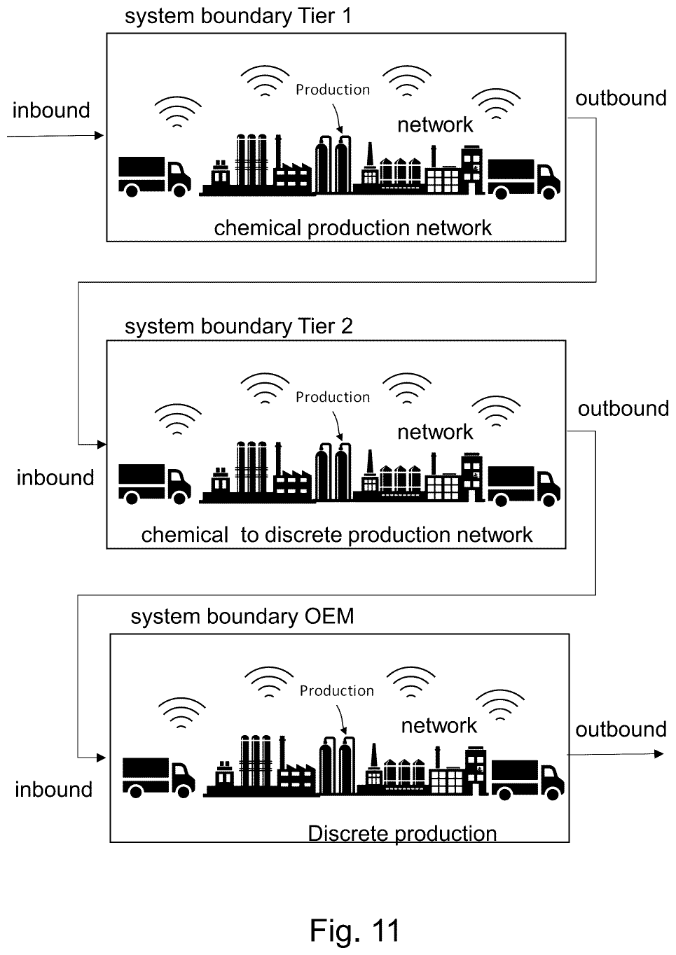

FIG. 11 illustrates an example of a method for producing at least one chemical product associated with at least one environmental attribute for a supply chain.

The example of FIG. 11 illustrates two tiers and an original equipment manufacturer. Tier 1 may be a chemical producer operating the chemical production network. The chemical production network may be associated with a system boundary. The system boundary may signify the physical boundary of the chemical production network. The input material(s) entering the chemical production network at any stage of the production network or the system boundary of the chemical production network may signify entry points to the chemical production network. On entry of input material(s) associated with one or more environmental attributes the environmental attribute(s) may be decoupled from the physical material flow of the input material(s) through the chemical production network. Such separation of the physical input material and the virtual environmental attribute may be provided by the method for registering at least one input material associated with at least one environmental attribute as described herein and as for example illustrated in FIGS. 4a (inbound). Chemical product(s) may be produced from input material(s) by the chemical production network. At least one chemical product may be produced by the chemical production network and the chemical product may be linked to at least one environmental attribute as described herein and illustrated in FIG. 4b (outbound).

The chemical product(s) associated with at least one environmental attribute as produced by the chemical production network may be provided to the next tier. In the illustrated example tier 2 may be a production network producing discrete products from chemical product(s) or chemical products. Discrete products may be any products associated with a distinct physical units. Discrete manufacturing in contrast to process manufacturing use such discrete products to assemble other discrete products. Chemical production in contrast uses process manufacturing where input material(s) are mixed and chemically converted to chemical output product(s). Such output product(s) are chemical product(s) that may be transferred in discrete units, such as containers or wreath. The chemical product(s) are such are however no discrete product(s). The chemical to discrete production network may be associated with a system boundary as described above. Similar to the chemical production network the chemical to discrete production network may on inbound register input material(s) associated with at least one environmental attribute, which may be the chemical product(s) of the chemical production network as described herein or illustrated in FIGS. 4a (inbound). Discrete output product(s) may be produced by the chemical to discrete production network. At least one discrete output product may be linked to at least one environmental attribute (outbound).

The discrete product(s) associated with at least one environmental attribute as produced by the chemical to discrete production network may be provided to an original equipment manufacturer producing end products. As described herein and in the context of FIG. 4a, the discrete product(s) and associated environmental attribute(s) may be decoupled on registration (inbound). As described herein and illustrated in FIGS. 4b after decoupling the environmental attribute may be linked to the end product as described herein and illustrated in the context of FIG. 4b (outbound).

The one or more environmental attribute(s) assigned to the chemical product identifier may be used to provide at least one discrete product identifier relating at least one discrete product or at least one end product identifier specifying at least one end product of a product supply chain to the one or more environmental attribute(s). The chemical product associated with one or more environmental attribute(s) may be used to produce at least one discrete product or at least one end product of a product supply chain associated with the one or more environmental attribute(s).

This way the environmental attributes associated with any input material(s) may be tracked through the value chain up to the end product. The methods, apparatuses and systems described herein enable transparency from early stages of the value chain on chemical product level to end stage of the value chain on end product level. Through linking of physical material and environmental attribute environmentally friendly products and more sustainable production can be made transparent and tracked.

1-15. (canceled)

16. A method for producing at least one chemical product associated with one or more environmental attributes, wherein the chemical product is produced by a chemical production network wherein the chemical production network chemically converts input materials via chemical intermediates to chemical products that exit the chemical production network, the method comprising:
providing multiple input materials associated with one or more environmental attribute(s) to the chemical production network,
providing at least one balancing account associated with one or more environmental attribute(s) of the multiple input materials provided to the chemical production network,
producing the at least one chemical product,
providing an identifier associated with the chemical product, and
assigning at least one environmental attribute of the multiple input materials from the at least one balancing account associated with the respective environmental attribute(s) to the at least one chemical product, wherein the at least one environmental attribute from the at least one balancing account associated with the respective environmental attribute(s) is assigned to the identifier associated with the chemical product, wherein the identifier associated with the chemical product includes a chemical product identifier relating to a chemical product specification and one or more environmental attribute identifier(s) relating to one or more environmental attribute(s) of the multiple input materials, wherein for assignment one or more target environmental attribute(s) for the chemical product are provided and depending on the provided one or more target environmental attribute(s) one or more environmental attribute identifier(s) are linked to the chemical product identifier, and wherein the chemical product identifier is associated with the physical entity of the chemical product, wherein the chemical product identifier is a virtual identifier uniquely linked to the chemical product.

17. The method of claim 16, wherein for assignment the one or more environmental attribute identifier(s) are linked to the chemical product identifier.

18. The method of claim 16, wherein the one or more environmental attribute identifier(s) relate to one environmental attribute or a combination of environmental attribute(s).

19. The method of claim 16, wherein the one or more environmental attribute(s) refer to a property related to the environmental impact, wherein the property is a property of the input material(s).

20. The method of claim 16, wherein the chemical production network includes connected, interconnected and/or non-connected production chains, wherein the chemical production network produces from input materials multiple intermediates and from intermediates chemical products.

21. The method of claim 16, wherein the chemical production network comprises one or more entry point(s) at which input materials are provided to the chemical production network.

22. The method of claim 16, wherein the chemical product identifier and the environmental attribute identifier(s) are unique identifiers for the specific chemical product and the environmental attribute(s) respectively.

23. The method of claim 16, the chemical product identifier is generated for each chemical product produced by the chemical production network, wherein the chemical product identifier is selected from a set of pre-generated chemical product identifiers for chemical products produced by the chemical production network, wherein the one or more environmental attribute identifier(s) are generated for environmental attribute(s) available to the chemical production network, wherein the one or more environmental attribute identifier(s) are related to input materials entering the chemical production network, wherein the environmental attribute identifier(s) are selected from a set of pre-generated environmental attribute identifiers for environmental attributes available to the chemical production network.

24. The method of claim 16, wherein for assignment the chemical product identifier is generated or selected based on the chemical product.

25. The method of claim 16, wherein for assignment the environmental attribute identifier(s) are generated or selected based on the one or more environmental attribute(s) to be assigned from the input materials to the chemical product.

26. The method of claim 16, wherein the chemical product identifier is linked to a physical identifier attached to the chemical product and/or wherein the chemical product identifier is linked to a batch number and/or an order number associated with the chemical product.

27. A method for assigning at least one environmental attribute to at least one chemical product produced by a chemical production network, wherein the chemical production network chemically converts input materials via chemical intermediates to chemical products that exit the chemical production network, the method comprising:
providing at least one balancing account and an account balance associated with one or more environmental attribute(s) from multiple input materials provided to the chemical production network,
providing an identifier associated with the chemical product, and
assigning at least one environmental attribute from the at least one balancing account associated with the respective environmental attribute(s) to the at least one chemical product, wherein the at least one environmental attribute from the at least one balancing account associated with the respective environmental attribute(s) is assigned to the identifier, wherein the identifier associated with the chemical product includes a chemical product identifier relating to a chemical product specification and one or more environmental attribute identifier(s) relating to one or more environmental attribute(s) of the multiple input materials, wherein for assignment one or more target environmental attribute(s) for the chemical product are provided and depending on the provided one or more target environmental attribute(s) one or more environmental attribute identifier(s) are linked to the chemical product identifier, and wherein the chemical product identifier is associated with the physical entity of the chemical product, wherein the chemical product identifier is a virtual identifier uniquely linked to the specific chemical product.

28. A system for producing at least one chemical product associated with one or more environmental attributes, wherein the chemical product is produced by a chemical production network, wherein the chemical production network chemically converts input materials via chemical intermediates to chemical products that exit the chemical production network, the system comprising:
a chemical production network configured to produce at least one chemical product based on multiple input materials provided to the chemical production network, wherein the multiple input materials are associated with one or more environmental attribute(s);
an accounting system configured to provide at least one balancing account associated with one or more environmental attribute(s) of the multiple input materials provided to the chemical production network,
an identifier provider configured to provide an identifier associated with the chemical product, and
an assignor configured to assign at least one environmental attribute from the at least one balancing account associated with the respective environmental attribute(s) to the at least one chemical product, wherein the at least one environmental attribute from the balancing account associated with the respective environmental attribute(s) is assigned to the identifier associated with the chemical product, wherein the identifier associated with the chemical product includes a chemical product identifier relating to a chemical product specification and one or more environmental attribute identifier(s) relating to one or more environmental attribute(s) of the multiple input materials, wherein for assignment one or more target environmental attribute(s) for the chemical product are provided and depending on the provided one or more target environmental attribute(s) one or more environmental attribute identifier(s) are linked to the chemical product identifier, wherein the chemical product identifier is associated with the physical entity of the chemical product, and wherein the chemical product identifier is a virtual identifier uniquely linked to the specific chemical product.

29. An apparatus for assigning at least one environmental attribute to at least one chemical product produced by a chemical production network, wherein the chemical production network chemically converts input materials via chemical intermediates to chemical products that exit the chemical production network, the apparatus comprising:
an accounting system configured to provide at least one balancing account including an account balance associated with one or more environmental attribute(s) from multiple input materials provided to the chemical production network,
an identifier provider configured to provide an identifier associated with the chemical product, and
an assignor configured to assign at least one environmental attribute from the at least one balancing account associated with the respective environmental attribute(s) to the at least one chemical product, wherein the at least one environmental attribute from the balancing account associated with the respective environmental attribute(s) is assigned to the identifier associated with the chemical product, wherein the identifier associated with the chemical product includes a chemical product identifier relating to a chemical product specification and one or more environmental attribute identifier(s) relating to one or more environmental attribute(s) of the multiple input materials, wherein for assignment one or more target environmental attribute(s) for the chemical product are provided and depending on the provided one or more target environmental attribute(s) one or more environmental attribute identifier(s) are linked to the chemical product identifier, wherein the chemical product identifier is associated with the physical entity of the chemical product, and wherein the chemical product identifier is a virtual identifier uniquely linked to the specific chemical product.

30. A method to produce at least one discrete product or at least one end product of a product supply chain associated with the one or more environmental attribute(s) comprising using the chemical product associated with one or more environmental attribute(s) as provided by the method of claim 16.
